(19) United States
(12) Patent Application Publication
(10) Pub. No.: US 2002/0115986 A1
(43) Pub. Date: Aug. 22, 2002

(54) Endovascular medical devices and techniques for delivering therapeutic agents

(76) Inventor: John H. Shadduck, Tiburon, CA (US)

Correspondence Address: Norman R. Klivans, SKJERVEN MORRILL MacPHERSON LLP, 25 Metro Drive, Suite 700, San Jose, CA, 95110-1349, US

(21) Appl. No.: 09/789,860

(22) Filed: Feb. 20, 2001

Related U.S. Application Data
(60) Provisional application No. 60/231,678, filed on Sep. 11, 2000.

(51) Int. Cl. A61F 2/06

(52) U.S. Cl. 604/891.1; 623/1.42; 607/156

(57) ABSTRACT

A system for controlled and automated delivery of pharmacological agents from an implantable endovascular medical device, for example to deliver an antiretroviral regimen to an HIV-infected pregnant mother to prevent transmission of the virus to the child in utero. A first component of the system is an implantable member dimensioned for insertion into a patient's vasculature, either natural or synthetic that delivers energy across a therapeutic interface between the member and body media, such as blood flow and the wall of the vasculature. The implanted member carries a helical coil in its wall that is responsive to selected electromagnetic energy radiation. A second component of the system is a portable transmitter system located at the exterior of the patient's body to transmit electromagnetic energy signals to the implanted member which is received by the helical coil. Microcircuitry coupled to the helical coil within the wall of the implanted member is coupled electrically to anode and cathode surface portions of the implanted member to release or expose pharmacological agents at the energy delivery interface of the member. The portable transmitter system is programmable to allow controlled delivery of the pharmacological agents from at least one surface layer carrying the agent to the endovascular media.

### CROSS-REFERENCE TO RELATED APPLICATIONS

This application is related to and claims priority to Provisional U.S. Patent Application Ser. No. 60/231,678 filed Sep. 11, 2000 (Docket No. S-QS-001) having the same title as this disclosure. This application also is related to U.S. patent application Ser. No. 09/715,370 filed Nov. 17, 2000 (Docket No. S-QS-005) titled “Intraluminal Prosthetic Device for Inhibiting Restenosis” which claims priority from Provisional U.S. Patent Application Ser. No. 60/166,724 filed Nov. 22, 1999 (Docket No. S-QS-004) having the same title. All of the above applications are incorporated herein by this reference.

### BACKGROUND OF THE INVENTION

1. Field of the Invention

The present invention relates to an intraluminal medical device adapted for implantation in a patient's vasculature, either natural or synthetic, for delivering energy to a therapeutic interface between the medical device and body media, such as blood flow and the wall of the vasculature for various therapeutic purposes such as pharmacological agent delivery. In an exemplary embodiment, the implantable medical device of the present invention provides a system for controlled delivery of antiretrovirals (e.g., AZT, nevirapine) to prevent HIV-infected pregnant mothers from passing the virus to an unborn child in utero.

2. Description of Background Art

Several factors prevent the widespread use of drug regimens for treating HIV-infected patients in less developed countries. One factor has been the high price of AIDS drugs, but leading pharmaceutical companies have collectively agreed to reduce the prices of antiretrovirals for countries in need. One company has agreed to provide nevirapine to developing economies at no cost for the next five years. Beyond the issue of drug cost is the issue of the required health care infrastructure for effectively treating a HIV patient population. The medications need to be handled and distributed properly, with assurance that patients follow drug regimens carefully. This aspect of health care delivery for treating HIV-infected patients with antiretrovirals is fraught with difficulties in a developing economy. For example, some antiretrovirals are intended to be taken orally on an empty stomach, some drugs need to be taken with meals and some antiretrovirals must be stored in a refrigerator. If the patient misses the required doses of an antiretroviral agent, the virus may become drug-resistant.

An even more complex problem for treating HIV-infected patients relates to individual patient monitoring. Longer-term success of an antiretroviral drug regimen depends upon monitoring the viral resistance of an individual patient which is in flux. It is well known that HIV mutates rapidly, and therefore a particular antiretroviral or set of antiretrovirals will not work for any individual patient forever. Patients must be monitored so they can switch among drug regimens when a viral strain becomes resistant. For example, current tests can measure the number of a patient's CD4 cells, the immune cells that are targeted by HIV. Alternatively, assays may be made of the patient's viral load-the concentration of HIV cells in a patient's blood. Such tests for monitoring viral resistance or viral load require frequent visits to clinicians and typically are expensive. The tests may be complex and may require skilled laboratory technicians as well as modem labs.

Future monitoring means are in trials. For example, physicians can now test the genotype—the genetic makeup—of a patient's particular strain of HIV virus to determine which antiretroviral may work best. Such genotyping involves a search of a HIV sample for known mutations that render the virus resistant to pharmacological agents based on database reference. Phenotyping is another form of test that is being developed that measures the HIV strain's susceptibility to various pharmacological agents. The current state of phenotyping requires growing the virus in the presence of a drug panel to determine which drug works optimally.

New methods for delivering pharmacological agents are needed for treating HIVinfected patients, particularly in developing economies. Preferably, such new methods would require less need for continuous intervention by health care personnel. Further, such new methods preferably would allow for programmable modulation of a drug delivery regimen in response to monitoring a patient's condition.

### SUMMARY

It is believed that certain sub-sets of the HIV-infected patient populations, particularly in developing economies, can be offered treatment with antiretrovirals and other drugs that can be delivered intravenously from an implantable medical device. This disclosure will use, as an example, one of the most important uses of an antiretroviral drug regimen which relates to the prevention of HIV transmission from a pregnant mother to her unborn child.

This disclosure is directed to (i) advanced methods for controlled release of pharmacological agents from an implantable device that requires only periodic intervention by health care personnel, for example, to reliably deliver antiretrovirals during a women's pregnancy; and (ii) implantable medical therapeutic devices that provide methods for modulating drug delivery with non-contact electromagnetic energy delivery from a programmable source at the exterior of the patient's body.

More in particular, the present invention is directed to an implantable endovascular medical device for application of energy at an interface of the medical device and endovascular media to thereby deliver or release a pharmacological agent. The elongate tubular medical device is placed within the patient's natural or artificial vasculature, or can be a portion of a synthetic vascular graft. For example, the elongate implant device may be inserted into a subcutaneous synthetic arteriovenous graft placed in a patient's forearm as is common in dialysis patients. The surfaces of the implanted tubular device around a central passageway define the energy delivery interface. Further, in one embodiment, the surface of the device carries multiple thin layers of pharmacological agents.

The wall of the tubular implantable device carries at least one conductive element that is responsive to electromagnetic energy transmitted from an external source, such as a portable transmitter carried at the skin surface over the implanted device. Typically, a helical coil is provided in the wall of the implant to function as an antenna component of the energy receiving circuitry, together with a capacitor system for transient energy storage. In use, radio frequency energy is transmitted from the external source to the implanted device which then converts the received radio frequency energy into electrical potential applied to exposed anode and cathode conductive surfaces of the device—the energy delivery interface. This electrical potential causes the transfer of electrons across the interface thereby applying energy to the endovascular media. The applied electrical potential is rather low—typically under three to five volts—although not so limited. In one embodiment, the electrical potential causes an electrochemical reaction at the surface of the device to remove very thin layers of conductive material to expose the pharmacological agent. In such an embodiment the surface of the device is coated with, for instance, alternating layers of a conductive material and a pharmacological agent. Such electrochemical removal of micron-thick surface layers thereby allows controlled delivery of the pharmacological agent directly into the patient's bloodstream. By successive removal of very thin surface layers as described above, signaled by a programmable external source, the patient can receive periodic doses of the pharmacological agent carried by the implant.

In using the system to treat an HIV-infected pregnant mother to prevent HIV transmission to an unborn child, the implant can carry a known antiretroviral agent within a synthetic vascular graft. A preferred antiretroviral agent for this purpose is AZT, which in an oral regimen requires twice-per-day doses for the last two months of pregnancy and higher doses every three hours during labor. The programmable feature of the present invention allows for suitable smaller doses at the requisite intervals since the agent is delivered intravenously. The periodic doses could be delivered reliably without intervention of health care specialists, once the implant was in place. The dosimetry could be adjusted after period monitoring during pregnancy. The higher doses required during labor also could be programmed into the external transmitter. Another antiretroviral agent that can be delivered for preventing the transmission of HIV to an unborn child is nevirapin (Viramun) which also may be a candidate for use with the implant system of the present invention.

The implant of the present invention also may carry drugs (agents) for treating opportunistic infections that affect HIV patients. For example, Fluconazole or Diflucan are known to be effective agents in treating cryptococcal meningitis, candidiasis and other infections of the mouth and esophagus that can be delivered by the implant of the present invention.

An implant also may carry antiretrovirals and other pharmacological agents used for long-term treatment of HIV patients selected from the following classes: Nucleoside analogs (e.g., lamivudine (Epivir); zalicatabine (Hivid); zidovudine (Retrovir); didanosine (Videx); stavudine (Zerit); abacavir (Ziagen)); Non-Nucleosides (e.g., delavirdine (Rescriptor); etavirenz (Sustiva); nevirapine (Viramune)); Adjunctive anti-retrovirals (e.g., Hydroxurea (Hydrea)); Nucleotides (e.g., adelovir (Preveon)); and protease inhibitors (e.g., amprenavir (Agenerase); saquinavir (Fortovase); indinivir sulfate (Crixivan); ritonavir (Norvir); nelfinavir mesylate (Viracept); 3TC (epirvir).

Another embodiment of the invention utilizes the energy responsive circuitry of the implant device to create a voltage differential at the energy delivery interface to cause the migration of a charged pharmacological agent from a microporous polymer surface coating of the device.

Another embodiment of the invention can deliver pharmacological agents from an exterior surface of the implanted device to the wall of a natural blood vessel to prevent vessel inflammation or proliferation of cells about surfaces of the device.

The system of the present invention advantageously provides an endovascular implant that provides an energy delivery interface for delivering energy to endovascular media that is relayed from an external electromagnetic transmission source.

The system of the invention provides an endovascular implant that provides for controlled release of pharmacological agents.

The system of the invention advantageously provides an endovascular implant that allows for controlled release of antiretrovirals in treating HIV patients such as pregnant women to prevent transmission of HIV to unborn children.

The system of the invention provides a programmable system for in vivo modulation of dosimetry in the release of pharmacological agents from an endovascular implant.

The system of the invention provides a endovascular implant that carries an antenna that can receive electromagnetic energy from an external transceiver.

The system of the invention provides a method for electrochemical removal of conductive surface layers of an endovascular implant to expose pharmacological agents.

The system of the invention provides a method causing the migration of a charged pharmacological agent from a polymer surface coating of an endovascular implant.

The system of the invention provides a method for electrochemical removal of physiologic surface accumulations on an endovascular implant.

### BRIEF DESCRIPTION OF THE DRAWINGS

Other objects and advantages of the present invention will be understood by reference to the following detailed description of the invention when considered in combination with the accompanying Figures, in which like reference numerals throughout are used to identify like components throughout this disclosure.

FIG. 1 is a schematic view of a patient's arm and a synthetic vascular graft that carries the implanted endovascular therapeutic device of the present invention and an external transceiver; the system, for example, adapted for the controlled release of a pharmacological agents such as an antiretroviral in a HIV treatment.

FIG. 2A is a cut-away perspective view of a Type “A” implant device in accordance with the present invention deployed within a synthetic vascular graft.

FIG. 2B is an enlarged cut-away view of a portion of the Type “A” implanted device of FIG. 2A taken along line 2B-2B of FIG. 2A rotated 90° together with the external transceiver.

FIGS. 3A-3B are perspective views of an alternative Type “A” device in accordance with the present invention; FIG. 3A being the implanted therapeutic device in a deployed or expanded position; FIG. 3B being the medical therapeutic device in a pre-deployed or non-expanded position.

FIG. 4 is a perspective view of a prior art endovascular prosthesis.

FIG. 5A is a cut-away view of a portion of the Type “A” implanted medical device of FIG. 2B.

FIG. 5B is an enlarged sectional view of the illustration of FIG. 5A taken along line 5B-5B of FIG. 5A.

FIG. 5C is a more greatly enlarged sectional view of the illustration of FIG. 5B taken along line 5C-5C of FIG. 5B.

FIG. 6 is an enlarged cut-away view of a surface portion of an alternative embodiment of a Type “A” implanted device.

FIG. 7 is a greatly enlarged cut-away view of a surface portion of a Type “B” device of the present invention.

### DETAILED DESCRIPTION OF THE INVENTION

I. Operational Principles Underlying Implantable Endovascular Medical Device

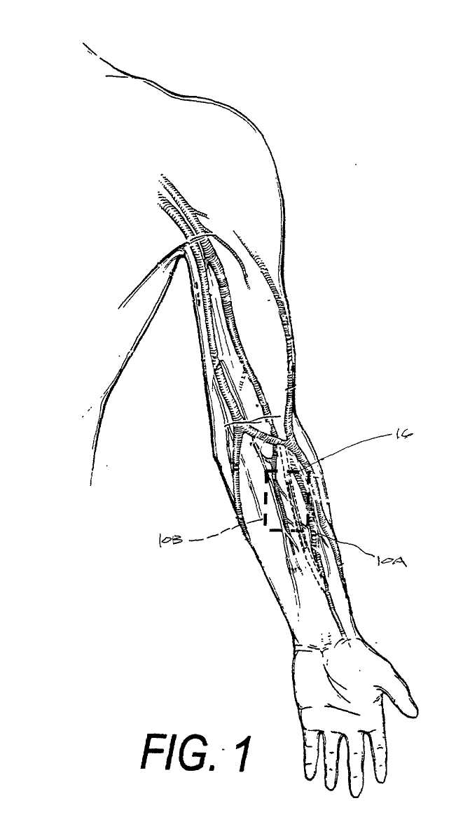

Several principles relating to the operation of an exemplary medical therapeutic system 5 will be described in the following sections. The system includes an implanted endovascular device 10A and an electromagnetic transmitter-receiver system 10B that is exterior to the patient's body. The description and techniques connected with FIG. 1 are described for exemplary purposes in treating patients infected with HIV, however, the techniques are not intended to be limited in their application to HIV patients.

A. Operational Principle: Endovascular Electrolytic Processes.

In U.S. patent application Ser. No. 09/494,828 (incorporated herein by reference) an endoluminal cardiovascular therapy was disclosed with a treatment device that delivered very low level DC electrical current to blood flow in proximity to a working surface of the treatment device to cause electrolysis. Such electrolysis at the working surface caused the formation of gas products at the working surface by energy delivery across the interface between working surface and the electrolyte (i.e., blood) which thereafter was utilized to enhance energy delivery to an endovascular occlusion. The delivery of energy at the interface between the working surface and endoluminal media, wherein such transfer of energy is defined generally as the movement or exchange of electrons, an electrochemical energy event, an electrical current flow or a transient voltage differential, essentially causes a renewal of the conductive surface structure or cleansing of the working surface. Such a renewal of the conductive working surface is of interest for maintaining a conductive surface or an electrode surface in a suitable condition for delivering energy to endoluminal media. The disclosure of the utility of such an electrochemical process at an endovascular site in Ser. No. 09/494,828 related to the working end of a catheter, in which electrical energy was delivered to and across the working surface by leads in the catheter that are coupled to an electrical source. In U.S. patent application Ser. No. 09/715,370 filed Nov. 17, 2000 (Docket No. S-QS-005) titled “Intraluminal Prosthetic Device for Inhibiting Restenosis” (claiming priority from Provisional U.S. Patent Application Ser. No. 60/166,724 filed Nov. 22, 1999 (Docket No. S-QS-004)), another form of endovascular treatment system comprising an implantable device was disclosed that utilized a non-contact method for delivering energy to the implanted device via a transmitter system, and transient storage of electrical energy in the device. The purpose of the treatment device of Ser. No. 09/715,370 again was to deliver low level electrical energy across an interface between the working surface of the treatment device and the flow of blood about the working surface (i.e., blood comprising an electrolyte). The method of utilizing the energy delivery from the surface of the implanted device in co-pending Ser. No. 09/715,370 related to diagnostic methods as well as therapeutic methods.

It can be seen that the utility of techniques of such an electrochemical process at an intraluminal device's working surface disclosed in Ser. No. 09/494,828 can be combined with the non-contact energy delivery methods (and methods of transient storage of electrical energy) disclosed in Ser. No. 09/715,370. The combination of these technologies thus can enhance or facilitate the therapeutic methods of Ser. No. 09/15,370. Further, electrolysis at a conductive working surface created by non-contact energy provides additional methods for diagnosing and treating endovascular conditions.

By way of background, since this disclosure relates generally to uses of an electrochemical process about an interface of a medical therapeutic device and an endovascular environment, further explanation of electrolysis is useful. The electrolytic process was investigated by Michael Faraday (1791-1867) who worked out the so-called laws of electrolysis. The disclosures herein make use, indirectly, of two laws of electrolysis determined by Faraday. Faraday's First Law states “that the mass of a substance produced at an electrode during electrolysis is proportional to the quantity of electricity passed.” The Second Law states “that the quantity of electricity required to produce one mole of a substance from its ions is proportional to the charge on those ions.” Together, these two laws are summarized by two equations:

Q=I×t and Q=n×_{z}×F

wherein: Q, measured in coulombs (C), is the quantity of electricity, I measured in amps (A), is the current; t, measured m seconds (s), is the time; n is the number of moles of substance produced at the electrode; z is the charge on the ion; and, F is a constant, with a value of 96500 C mol⁻¹. Faraday discovered that the amount of deposition on an electrode is directly proportional to the current passing through an electrolytic cell. One faraday of current (96,500 coulombs) of current will deposit one gram equivalent mass of a substance. Two faradays of current will deposit two gram equivalent masses of substance and so on. A gram equivalent mass of a substance is equal to the formula mass of the substance divided by the number of moles of electrons passing through the cell.

In more generalized terms, electrolysis provides an electrochemical process by which electrical energy can be used to initiate or promote chemical reactions that occur at electrodes (i.e., conductive surfaces coupled to an electrical source) within an electrolyte. Electrolysis thus involves the passage of an electric current through a conducting solution such as an intraluminal fluid (i.e., an electrolyte) that is, in part, decomposed in the process. When a cathode (or negative electrode), and an anode (or positive electrode) are immersed within an electrolytic fluid, and a direct current source is connected to the electrodes, the positive ions migrate to the negative electrode and the negative ions migrate to the positive electrode. At the negative electrode each positive ion gains an electron and becomes neutral; at the positive electrode each negative ion gives up an electron and becomes neutral. The migration of ions through the electrolyte constitutes the electric current flowing from one electrode to the other. In other words, the anode revolves the oxidation process where species lose electrons that are deposited at the anode. A gain in electrons occurs at the cathode. The electrolyte (e.g., blood in this disclosure) is a solution that allows electrical current to move therethrough which is essential to any electrolytic process. While electrolysis often involves metals that are capable of being reduced at the cathode to metallic atoms that can then be deposited on the cathode surface, other compositions besides metals can undergo electrolysis. Water (e.g., in blood) can be electrolyzed producing hydrogen gas at the cathode and oxygen gas at the anode at a ratio of two volumes of hydrogen for every one volume of oxygen gas, which was disclosed for novel purposes in Ser. No. 09/494,828.

In the example of electrolysis in blood disclosed in Ser. No. 09/494,828, electrode portions of the working surface are exposed to the reactant, or blood flow about the working surface. When current is applied, a desired reaction occurs in two ways. Bubbles of gas will form at the surfaces of the electrodes as water decomposes into H₂ and O₂ gas (either a DC or AC current can provide sufficient current to cause decomposition of water). Decomposition of water is a redox reaction. That is, the oxidation reaction occurs at one electrode, and the reduction reaction at the other electrode. Water is oxidized at the anode and the reaction is:

2H₂O→O₂+4H⁺+4e⁻

Further, water is reduced at the cathode. The reaction is

4H₂O+4e⁻→2H₂+4OH⁻

In the decomposition reaction, the volume of 2H₂O produced is twice the volume of O₂:

2H₂O→2H₂+O₂

This electrochemical process, besides providing micro-scale volumes of hydrogen and oxygen from blood for methods disclosed in co-pending pending Ser. No. 09/494,828, can be used to remove or clean a sub-micron or micron-dimension thickness from a working surface of an endovascular medical therapeutic device for several purposes, described seriatim below after providing the parameters for the physical structure of an exemplary medical therapeutic device.

B. Operational Principle: Electromagnetic Energy Transceiver System

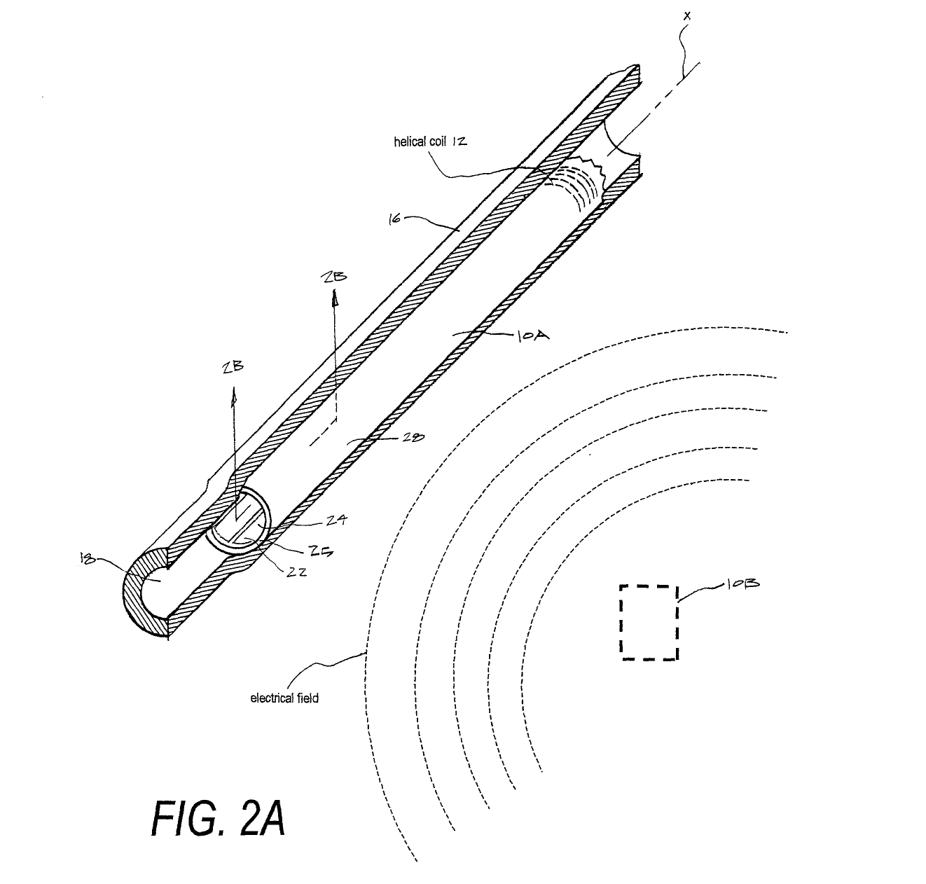

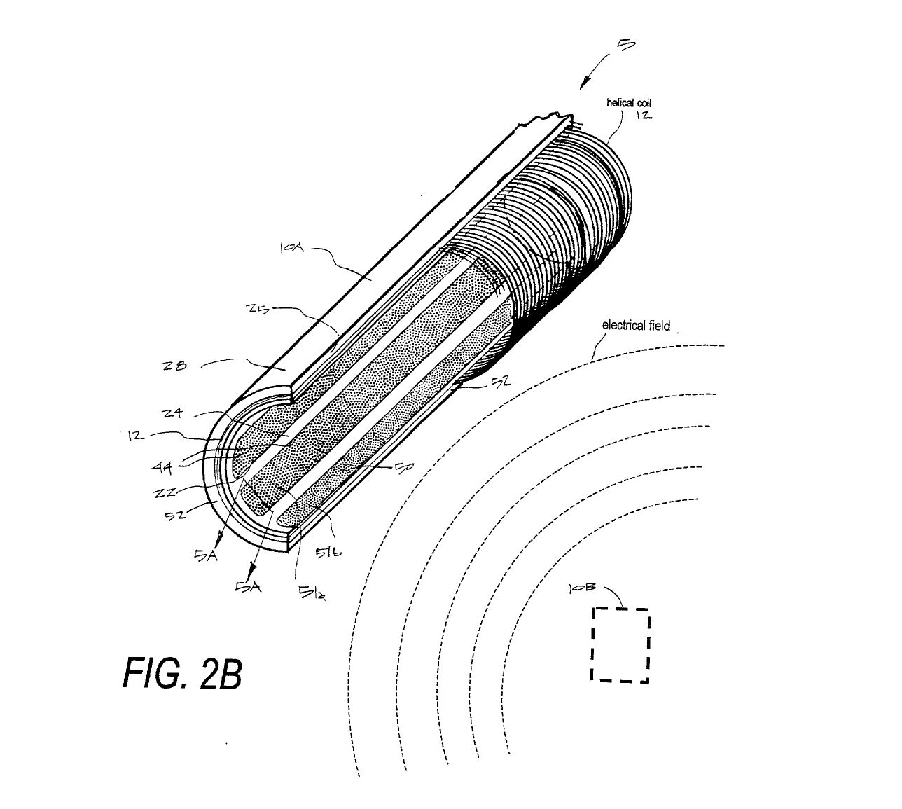

Systems were disclosed in Ser. No. 09/715,370 for the non-contact delivery of electromagnetic energy from a transmitter to an implanted endovascular device, together with transient storage of energy therein. It easily can be seen how the system of Ser. No. 09/715,370 can be used to deliver electric current to cause electrolysis as described above. Therefore, the bulk of that disclosure and its Figures will not be repeated here, for convenience. Certain components of the apparatus in Ser. No. 09/715,370 are shown in the Figures of this disclosure and use similar reference numerals such as the helical coil element indicated at 12 (see FIG. 2B). As described below, the circuitry of the system 5 is used to deliver electrical current to a certain conductive surface layer or layers of the endovascular device via a conductive lead portion.

II. Type “A” Endovascular Medical Therapeutic Device

The medical therapeutic device 10A in accordance with the present invention is adapted to be implanted in a patient's vasculature, whether synthetic or natural. Referring to FIG. 1, the location of one type of endovascular graft is shown, but it can be any similar graft, for example between the radial artery and cephalic vein or any other suitable arteriovenous loop. This type of graft is well known in the art and is common for dialysis access procedures. The actual graft location optionally can be in the forearm or upper arm, any of which is suitable for receiving the elongate implantable device of the present invention for delivery of pharmacological agents. A natural vessel may receive the implanted device 10A, but preferably for facilitating implantation of the medical device of the present invention, a PTFE (polytetraflouroethylene) graft indicated at 16 is used to receive this embodiment.

Referring to FIGS. 1 and 2A, this system embodiment (called herein Type “A” ) includes a first component comprising the exemplary implantable device 10A and a second component external to the patient's body comprising a cooperating portable transceiver 10B. FIGS. 2A & 2B show cut-away views of the implantable device 10A positioned in the lumen 18 of the synthetic graft 16. The device 10A can be any suitable length ranging from about 5 mm. to 200 mm. depending on the amount of pharmacological agent that it is required to carry. The implantable device can be of a rigid material in a short length or of a flexible material in a longer length. As will be described below, the surfaces of the device carry coatings or layers that carry pharmacological agents and therefore it is preferred that that walls of the device have substantial surface area to thereby allow for carrying greater amounts of a selected agent.

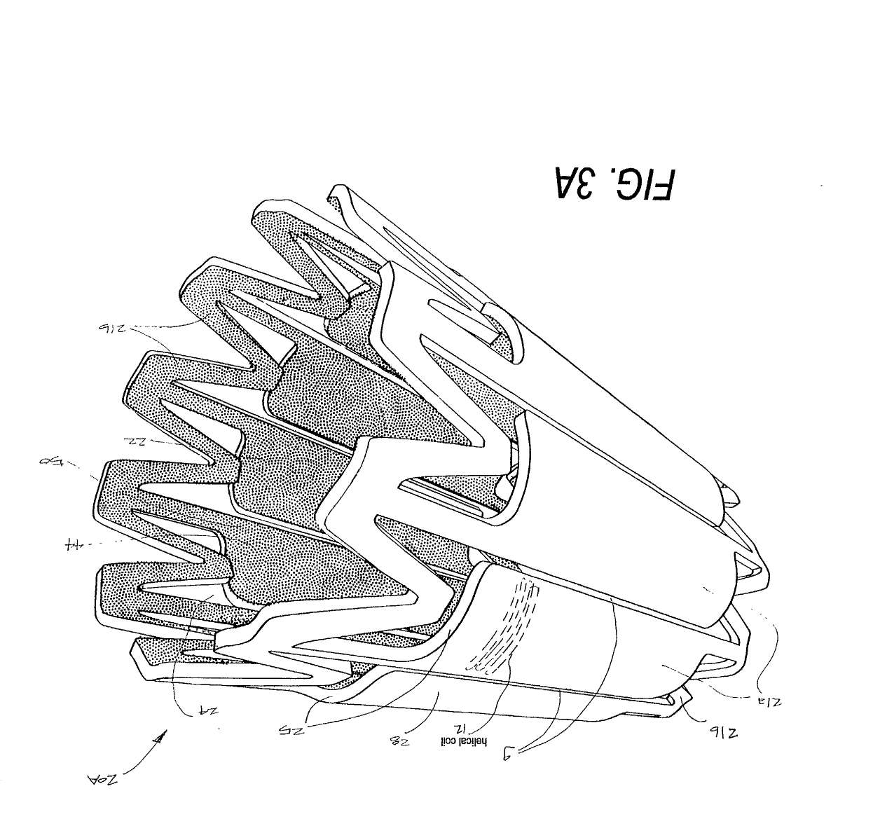

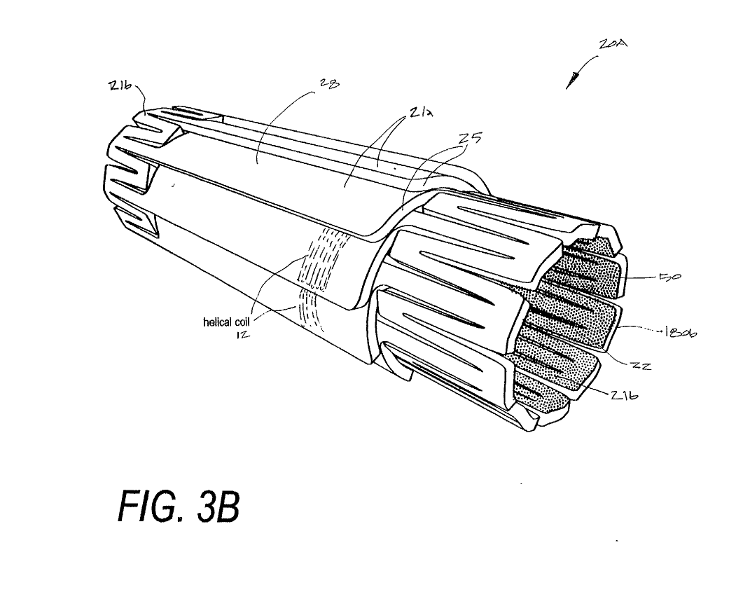

FIGS. 3A & 3B depict an alternative embodiment of a Type “A” device 20A that is adapted for a radially-expanded deployment within a lumen of a natural vessel. The implantable device 20A has wall substantially wide wall strut portions indicated at 21a together with bendable strut portions 21b that allow the device 20A to be deployed in a natural vessel by moving the device from its collapsed position (FIG. 3B) to its expanded position (FIG. 3A) by balloon-expansion means known in the art of stent deployment. It can be seen that device 20A of FIG. 3A provides wall portions having a substantial surface area for providing the therapeutic interface of the invention that would not be possible with a prior art stent as depicted in FIG. 4. The prior art stent of FIG. 4 is used as a prosthetic device and typically has thin cross-section struts 23. Such thin struts 23 provide little surface area for carrying pharmacological agents that interface with the flow of blood within the vessel lumen or that interface with the vessel wall.

The Type “A” devices 10A and 20A (FIGS. 2 & 3A) extend about axis x and define an exposed therapeutic surface or inner (first) energy delivery interface 22 around a central passageway 24. The device bodies generally define a wall portion indicated at 25 (see FIG. 2B) that surrounds passageway 24 which receives blood flow therethrough. The devices further define an exterior (second) energy delivery interface indicated at 28 that engages or contacts the vessel and therefore can be used to delivery agents to the walls of a natural vessel.

The following sections now will describe in more detail the construction of wall portion 25 of a Type “A” implantable such as shown in device 10A of FIG. 2B, but the description also can apply to the wall 25 of strut portion 21a of device 20A as illustrated in FIG. 3A.

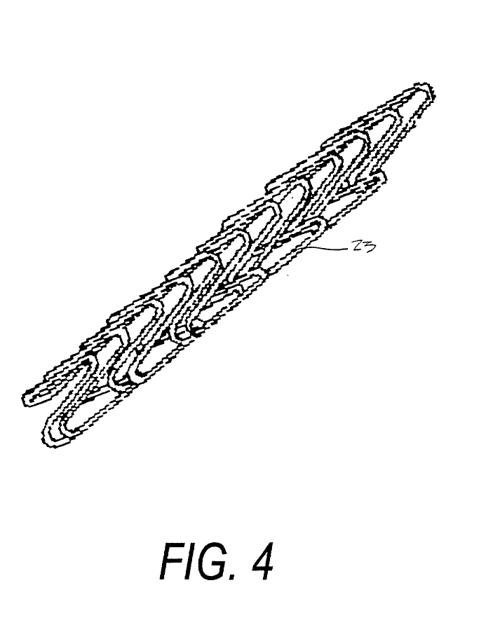

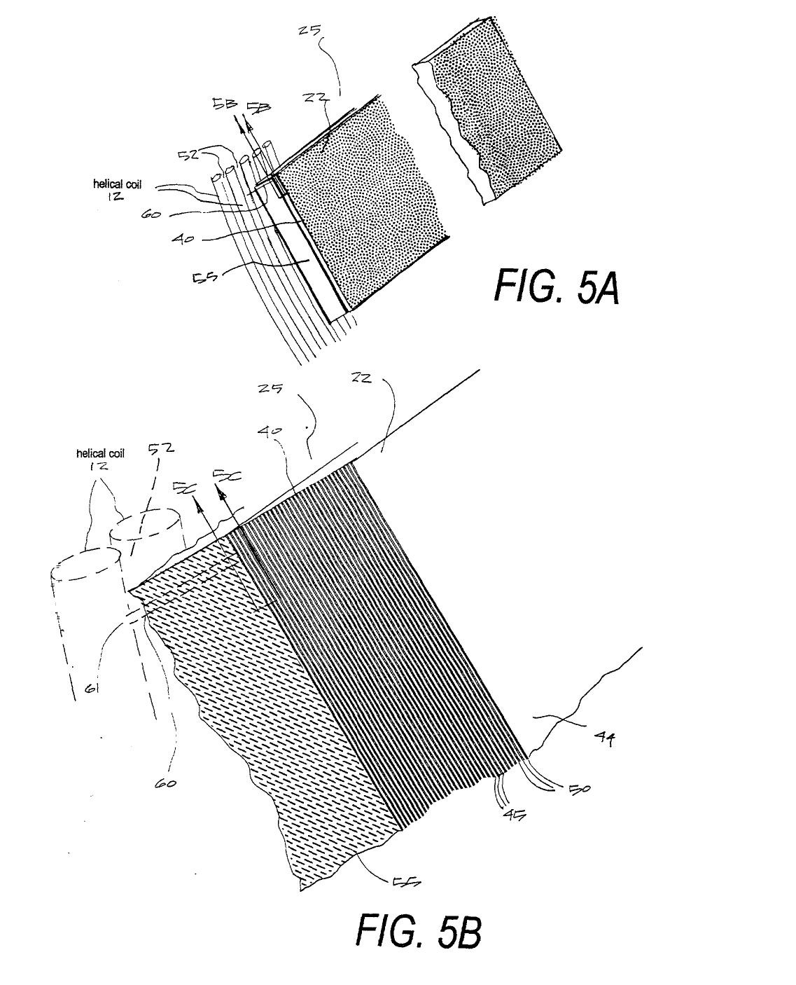

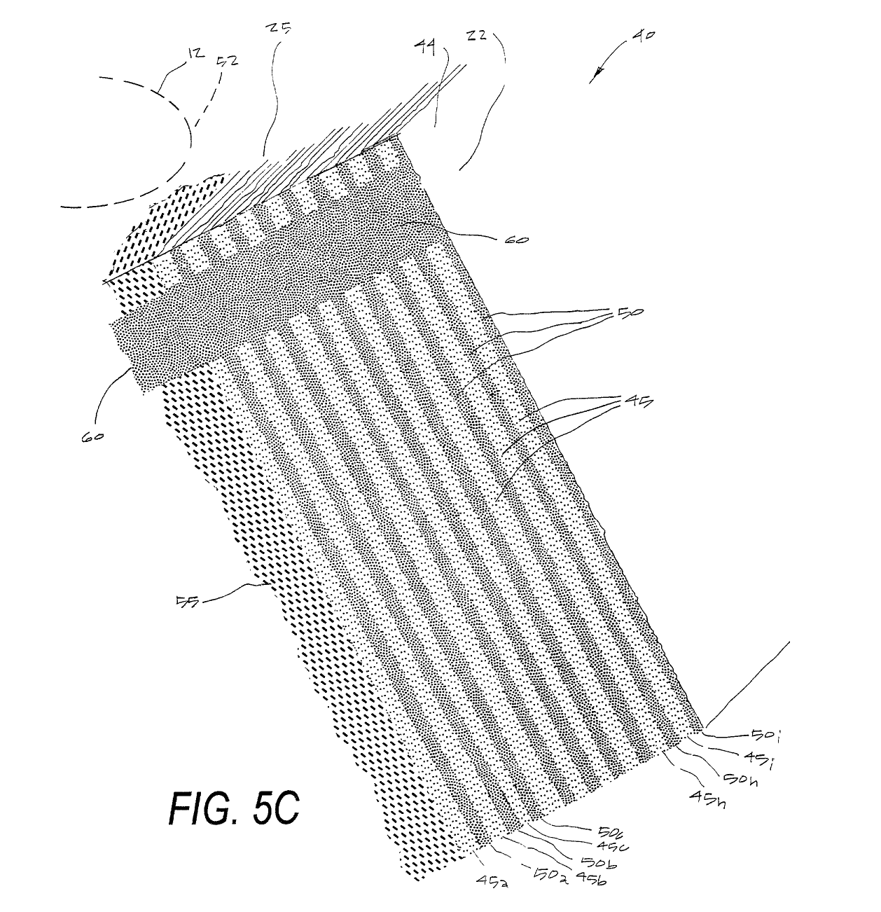

Now turning to FIGS. 2B, 5A & 5B, it can be seen that the energy delivery interface 22 or first surface of wall 25 carries a plurality of alternating layers of pharmacological agents and conductive compositions, indicated collectively at 40. FIGS. 5A & 5B are greatly enlarged views of a small portion of energy delivery interface 22. In FIG. 2B, the agent-carrying layers 40 are shown in a plurality of spaced apart surface portions (or cathode and anode portions) each having an exposed surface indicated at 44. This exposed surface will alternate between a therapeutically active layer 45 defined as carrying a pharmacological agent and an electrochemically reactive layer (or electrolytic layer) indicated at 50 that is provided for practicing a method in accordance with the invention. In this particular embodiment, the electrolyte layers 50 are separated spatially into cooperating spaced apart anode and cathode portions indicated at 51a and 51b, respectively (see FIG. 2B). It should be appreciated that the scope of the invention includes configuring anode and cathode portions is any suitable spaced apart arrangement or relationship. For example, the cooperating arrangements may be axial, circumferential, helical or simply a spaced apart random spot formation. The anode and cathode arrangement can have surface areas ranging from the micron-sized, to substantially the entire energy delivery interface 22 of the implantable device. That is, the anode could comprise practically the entire surface 22, and the cathode could comprise only a small exposed element at one or both ends of the device (not shown). Alternatively, the electrolytic layer 50 may simply include an exposed layer of gold that carries current flow that is exposed to blood flow, which thereby will exchange ions with NaCl in blood. Thus, the spaced apart longitudinal portions of the energy delivery interfaces of FIG. 2B are shown for convenience only. FIGS. 1A & 2B further show the transmitter/receiver portion 10B (or transceiver) of the system 5 with an electromagnetic field indicated at ef propagating in the direction of device 10A, all of which is disclosed in detail in Ser. No. 09/715,370 and is not repeated here.

Turning now to FIGS. 5A-5C, progressively enlarged cut-away views of a portion of device wall 25 are shown that illustrate the active layers 40 of the device that are made up of therapeutically active layers 45 and electrochemically reactive layers 50. Before further describing the active layers 40, it can be seen in FIG. 2B that device 10A (or device 20A of FIG. 3A) has a core structural element 52 of any suitable material such as a metal (stainless steel, nickel titanium, etc.) or a plastic. The core 52 is typically a structural component of the device that prevents the passageway 24 of the device from collapsing and is typically a flexible plastic in device 1A of FIG. 2A and a metal in the expandable device 20A of FIG. 3A. Of particular interest, FIG. 2B further shows that a helical conductive wire element 12, shown only in part, is provided in wall 25 and is insulated from core 52. In device 20A of FIG. 3A, each strut portion 21a has a helical conductive coil 12 similar to that shown in FIG. 2B.

Now returning to FIGS. 5A & 5B, it can be seen that the active layers 40 are deposited on an insulative substrate 55. The insulative substrate 55 can be any suitable material such as a plastic layer that electrically insulates helical coil or antenna 12 from the electrochemically reactive layers 50 as shown in detail in FIG. 5C.

FIG. 5C depicts a very small portion of the active layers 40, and more particularly shows a conductive layer 50 including the transient exposed surface portion 44 of the active layers 40 in the energy delivery interface 22 of device 10A. The conductive layer 50 is a biocompatible conductive material that can be reduced or electrochemically removed, such as gold. The therapeutic active layers 45 may be any pharmacological agent listed in detail above (e.g., antiretrovirals) that can be deposited on substrate 55 or in an active layer 50. The layers 45 and 50 are substantially thin, for example ranging down to the molecular dimension of the compositions and typically have a submicron thickness or a micron thickness. Thus, the scope of the invention covers conductive layers and therapeutic composition layers ranging from about 1 nm. in thickness to about 100 microns in thickness. The number of layers of therapeutic agent in the device can thus number in the hundreds or thousands or more, depending on the dimensions of the molecules and the quantity of agents that are require to be released into the blood stream to have therapeutic effect. It should be appreciated that each therapeutic layer may provide any dissolvable drug dose for a particular interval, such as one day, and the large number of layers thus may treat a patient for hundreds of days or more. The scope of the invention thus includes any number of layers 45 and 50 ranging from a single layer to about 5000 layers. The layers 45 and 50 may be deposited on the substrate 55 by several means known in the art, e.g., vapor deposition, electroless plating processes, plasma deposition and other thin-film application processes. As one example, SurModics, Inc. 9924 W. 74th St., Eden Prairie, Minn. 55344-3523 specializes in the deposition or application of therapeutic agents and other compositions as thin layer coatings on medical devices.

In FIG. 5C, the therapeutic agent layers 45 (collectively) are indicated at discrete layers 45a through 45i, which may be the first layers out of hundreds or thousands applied to substrate 55. Similarly, the initial conductive layers 50 on the substrate are indicated at discrete layers 50a through 50i. In order to deliver electrical current to the exposed surface conductive layer, for example layer 50i in FIG. 5C, a conductive portion or lead portion 60 extends through all layers 45 and 50. The proximal end 61 of this lead portion 60 extends to the core of device 10A and is coupled to the antenna 12 and circuitry systems therein that are described in detail in Ser. No. 09/715,370. In general, the helical coil or antenna 12 receives electromagnetic energy from the remote transceiver 10B and optionally causes transient storage of such energy and thereafter delivers current flow to the anode and cathode portions 51a and 51b of the surface 22 to thereby deliver energy to the exposed conductive layer 50i in FIG. 5C. Described in another way, the system causes a voltage differential at the energy delivery interface 22 between spaced apart anode and cathode surface areas.

One electrical lead 60 is shown in FIGS. 5A-5C to thereby carry current to, or from, the active layer 50i at an interface 44 to cause the movement of electrons across the energy delivery interface 22 of the device and endovascular media, in this case blood flow.

It can thus be understood that a first method in accordance with the invention is to cause an electrochemical reaction at energy delivery interface 22 for removing a surface conductive layer portion (i.e., a submicron thick layer of gold) from the exposed surface 44 of the device following a signal generated by transceiver 10B that is received by the helical coil 12 and other circuitry components within wall 25, as described in Ser. No. 09/715,370. An electric current is thereby caused at surfaces 51a and 51b as shown in FIG. 2A that are carried about surface 22 of the device, which causes a low level of current flow, and reaction within, conductive blood that flows in passageway 24 thereby causing electrolysis at interface 44. In a first mode of operation of the invention, the current flow is caused at a selected level ranging from about 0.1V to 5.0 V, and preferably between about 0.1 V to 3.0 V, to cause electrolysis and removal of gold layer 50i (see FIG. 5C) as the NaCl in blood reacts in various manners and exchanges ions with the gold layer causing its removal. This electrochemical event comprises a delivery of energy across the energy delivery interface to thereby provide a method for exposing a pharmacological agent layer to blood flow since removal of the gold layer 50i exposes the underlying layer 45i that carries the agent. The thickness dimension and surface dimension of the underlying pharmacological agent layer 45i is selected to provide a suitable dose for a particular time interval. Thus, in this first mode of operation, the device may be signaled to delivery agents (i.e., to remove a successive layer of conductive material to expose another layer or therapeutic dose) at selected pre-set time intervals, or at any manually signaled interval. In a preferred embodiment, the transceiver 10B is a programmable portable device that carries a transmitter component that can be patched to the patient's body overlying the implant. Thus, for example, a patient could be provided with regulated doses of an antiretroviral for the purposes described above in the section titled Summary of the Invention.

Of particular interest, it should be appreciated that one manner of practicing a therapeutic method of the invention comprises the step of electrochemically removing a thin conductive coating layer from the energy delivery surface 22 of the device to remove neointimal hyperplasia or any other cellular growths or other accumulation on the surface of the device that result from physiologic processes. This method can be practiced on an implanted device even if the device surface does not carry a pharmacological agent.

In another embodiment (not shown), the conductive composition within the energy delivery interface 22 can be somewhat dispersed within the layer that carries the pharmacological agent. In delivering energy to the surface of the implant as described above, the electrochemical removal of thin surface layers then would cause the release portions of the pharmacological agent into the bloodstream at the same time that portions of the conductive composition are electrochemically removed.

It should be appreciated that the drug delivery coatings or layers 45 described above can be customized further to release the pharmacological agent over time periods ranging from hours to months. The drug delivery layers 45, as well as conductive layers 50, can be further enhanced with agents to include delivery of antimicrobials. The layers also be combined with other treatment potential, for example, heparin and anti-adherence compositions on the same surfaces to provide added benefits to the device.

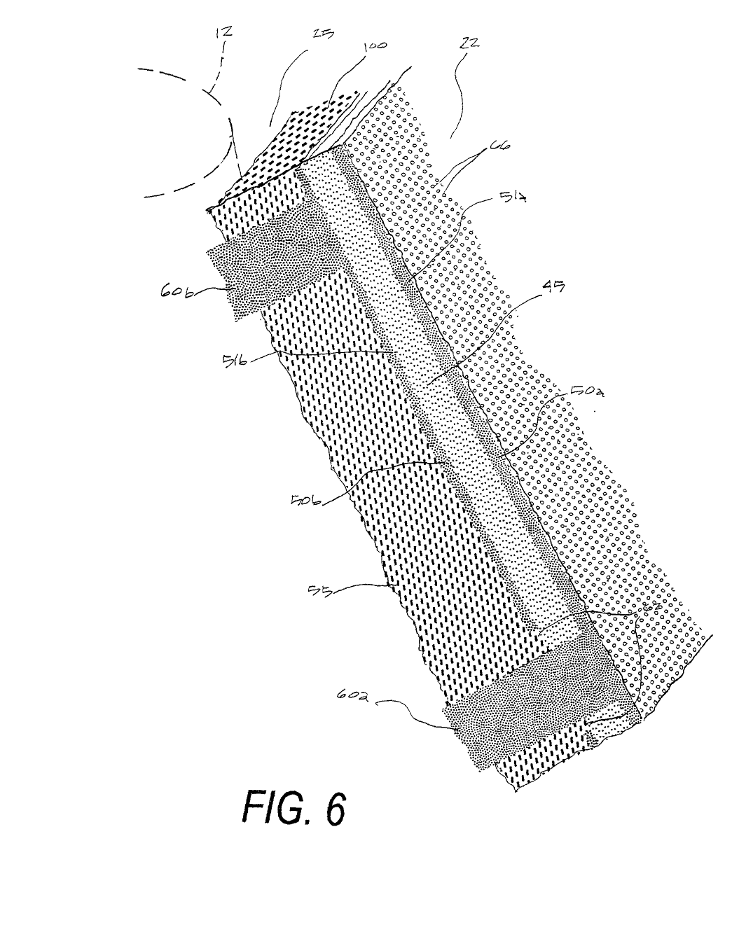

Referring to FIG. 6, another embodiment of Type “A” device is shown which uses the energy delivery means described above but delivers energy, or creates a voltage differential, across underlying and overlying spaced apart conductive layers that comprise an anode portion 51a and a cathode portion 51b. More in particular, the layer or reservoir strata carrying the pharmacological agent is indicated at 45 and is, for example, a porous non-conductive polymer layer that carries a fluid with the pharmacological agent therein. Also, the fluid carrying the agent has an electrical charge. The underlying conductive layer 50b that is laid down on the insulative substrate 55 is coupled to the coil and associated circuitry as described previously by lead portion 60b. This conductive layer may be any suitable thin metallic coating layer. The surface conductive layer 50a is a conductive polymer layer that has microporosities 66 extending therethrough. The porous polymer also may have a very thin metallic coating. This porous surface conductive layer is coupled to the coil and circuitry within wall 25 by lead portion 60a, that is insulated from conductive layer 50b at point 62 where the lead 60a passes through layer 50b.

Still referring to FIG. 6, the method of the invention can be described as a form of electrophoresis wherein the programmable transceiver 10B delivers electromagnetic energy to coil 12 and thereafter a voltage differential is created between the positive and negative conductive layers 50a and 50b. The charge carried by the fluid then will cause the fluid and pharmacological agent therein to migrate outwardly in response to the voltage differential and through porous polymer surface layer 50a into the endovascular media. When the charge is not applied, the agent remains in the interior reservoir layer 45. This embodiment thus provides a system for controlled release of drugs from an endovascular implant.

While FIG. 6 shows an exemplary energy delivery interface 22 at a first interior surface of an implant around a central passageway, it should be appreciated that the interface 22 also may be provided at the exterior (second) surface 28 of an implant, for example the device of FIG. 3A or a prior art device similar to that shown in FIG. 4. Such an energy delivery interface thus would be adapted to delivery pharmacological agents to a vessel wall in a controlled manner over any selected time interval. The agents that may be delivered may be any suitable drug and are fall into wide-ranging classes, such as antiproliferative agents (e.g., Sirolimus, troglitazone, Paclitaxel, Taxol), an anti-inflammatory agent (e.g., dexamethasone); any other antirestenotic or thrombolytic agent (e.g., heparin) or any antifibrogenic agent (e.g., FG-041 by FibroGen, Inc., 225 Gateway Blvd. So. San Francisco, Calif. 94080).

III. Type “B” Endovascular Medical Therapeutic Device

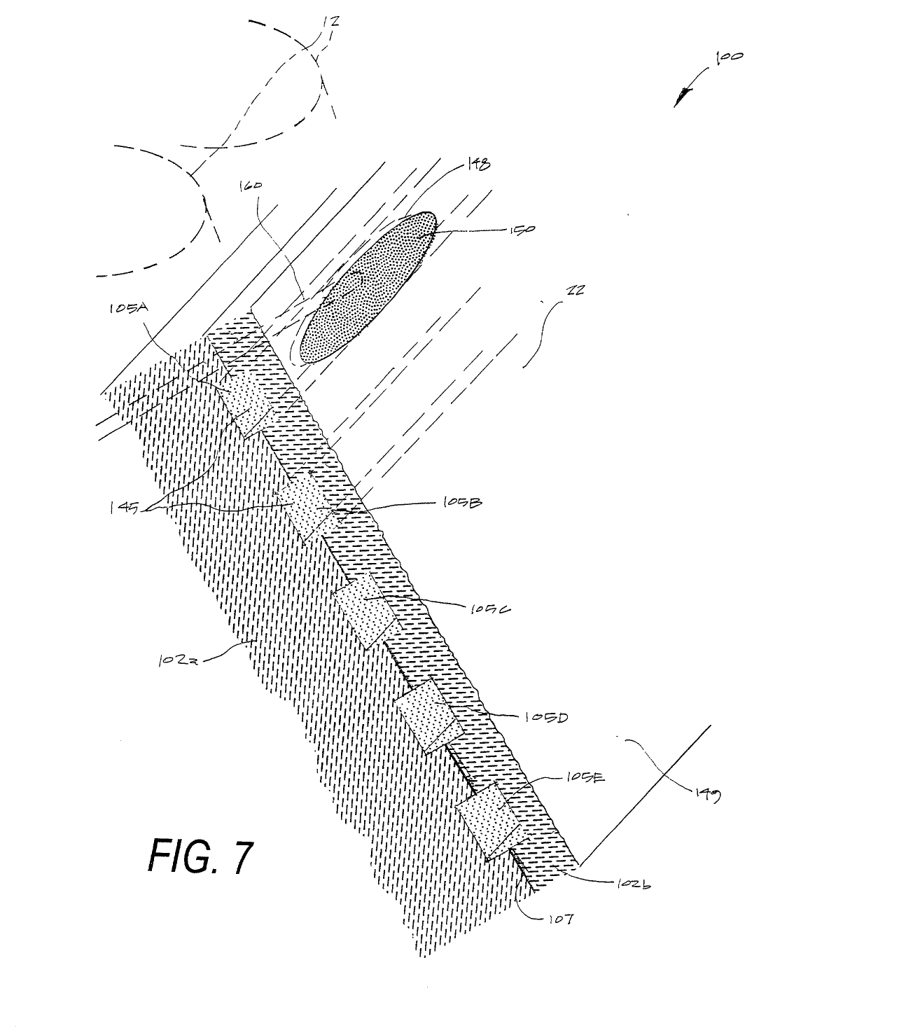

FIG. 7 illustrates a greatly enlarged portion of an exposed energy delivery interface 22 of another exemplary (called herein Type “B” ) device. The superstructure of the Type “B” device can be identical to that of the implantable devices described previously. The Type “B” device differs only in the manner of releasing or delivering the pharmacological agent. The wall 25 of a Type “B” device carries the helical coil 12 and circuitry as described above, but further includes micro-reservoirs for carrying the pharmacological agent plus electrochemical means for releasing the agents to endovascular media.

More in particular, FIG. 7 shows a small portion of a microfluidic chip assembly 100 fabricated (e.g., in silicon) conventionally by semi-conductor processing techniques that is carried in the wall 25 of the superstructure of the implantable device of FIG. 2B or FIG. 3A. FIG. 7 shows that first substrate layer 102a is bonded to a second substrate layer 102b. Either or both layers are etched to provide microchannels 105A through 105E shown in the schematic view of FIG. 7. Such channels for passage of microfluids are known in the art of semiconductor processing, with the pre-etched wafers bonded together at very high temperatures along line 107. Such microfluidic channels are known in so-called biological chips designed to facilitate identification of DNA sequences. The cross-sectional dimensions of such channels may range from about 1 μm to 100 μm and preferably is from about 5 μm to 50 μm with any suitable length. Other methods may be suitable for inexpensive fabrication of microchannels, such as creating a series of etchable photoresist ridges on a wafer surface. In such a process, a layer of suitable substrate is deposited over the ridges to form a layer. Thereafter, the photoresist is etched away or dissolved to provide the microchannels.

In FIG. 7, it can be seen that the pharmacological agent 145 is in a fluid form carried within microchannels 105A through 105E, wherein the number of channels may number in the hundreds or thousands. FIG. 7 further shows the electrochemical means for opening the reservoir to release agent 145. The pharmacological agents can be the same agents as described previously for similar purposes. Each channel has at least one media entrance port 148 in surface 149 that is covered with a thin layer of gold 150 or other conductive material that comprises a sacrificial covering over the port that can be reduced by electrolysis to open the port 148. Wire lead 160 extends from the conductive layer 150 to the circuitry of the device as described above. It can be easily understood that a successive number of reservoirs can be opened in sequence by directing current flow to selected ports to electrochemically remove the gold layer, generally as described previously. Thus, the mode of operation of the Type “B” device is the same as the Type “A” device, except that fluid reservoirs are exposed to blood flow rather than exposing the agents in successive layers.

The present invention is not limited to the specific embodiments described herein, but rather is defined by the scope of the appended claims. Specific features of the invention may be shown in some figures and not in others and this is for convenience only and any feature may be combined with another in accordance with the invention. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

I claim:

1. A method for energy application at an interface of a medical device and endovascular media, comprising the acts of:
(a) placing a medical device within a lumen of a patient's vasculature, the device having a surface defining an energy-delivery interface with endovascular media of said vasculature; and
(b) applying substantially low-level electrical potential at said interface by delivery of electromagnetic energy from a remote source, whereby said electrical potential causes electron transfer across said energy-delivery interface thereby applying energy to said media at said interface.

2. The method of claim 1 wherein said electrical potential at said interface is at a level ranging from about 0.01V to 5 V.

3. The method of claim 3 further comprising the act of providing a surface layer containing a pharmacological agent on said medical device.

4. The method of claim 1 further comprising the act of providing at least one helical coil within said medical device.

5. The method of claim 4 further comprising the act of providing a transmitter that transmits said electromagnetic energy to said medical device.

6. The method of claim 1 wherein said electrical potential electrochemically removes thin layer portions from said surface.

7. The method of claim 1 further comprising the act of providing a conductive polymer surface having microporosities on said medical device.

8. The method of claim 3 further comprising the act of providing a reservoir containing a pharmacological agent in said medical device with an electrochemically sacrificial layer over a port communicating with said reservoir.

9. The method of claim 6 wherein the electrochemical removal of thin layer portions includes removal of surface accumulations resulting from physiologic processes.

10. A medical device, comprising:
a member extending along an axis and having wall portions around a central passageway, the wall portions defining an engagement surface that engages endovascular media, the member dimensioned for implantation in a patient's vasculature;
at least one electrically conductive layer portion carried on said engagement surface;
at least one conductive element carried in said wall portion thereby being responsive to electromagnetic energy transmission; and
circuitry coupled to said conductive element thereby directing electromagnetic current to said at least one conductive layer.

11. The medical device of claim 10 wherein said conductive element is a helical coil.

12. The medical device of claim 10 further comprising at least one layer carrying a pharmacological agent proximate to said engagement surface.

13. The medical device of claim 10 wherein said engagement surface comprises a plurality of alternating layers of conductive compositions and pharmacological agents.

14. The medical device of claim 10 wherein said engagement surface comprises a microporous layer that carries a conductive composition.

15. The medical device of claim 10 further comprising a sacrificial conductive layer portion carried on said engagement surface over a port defined in said wall portion and that communicates with a reservoir in said wall portion.

16. The medical device of claim 10 wherein said member is moveable between a radially-collapsed state and a radially-expanded state.

17. A medical treatment method, comprising the acts of:
(a) implanting in a patient's vasculature a member that carries an antenna, the member carrying a pharmacological agent in a surface portion thereof;
(b) transmitting electromagnetic energy from the exterior of the patient's body to said antenna; and
(c) converting said electromagnetic energy received at said antenna into a voltage differential at the surface portion of the member thereby accelerating the release of the pharmacological agent from said surface portion.

18. The medical treatment method of claim 17 wherein the voltage differential causes the movement of electrons across the interface of said member and endovascular media.

19. The medical treatment method of claim 17 wherein the voltage differential causes the movement of the pharmacological agent toward the endovascular media from an interior of said surface portion.

20. An endovascular implant system comprising:
a member having a wall portion that defines a passageway extending therethrough, the member dimensioned for implantation in patient's vasculature;
at least one helically-wound element carried in said wall portion of said member comprising an antenna that is responsive to a selected electromagnetic energy transmission; and
an external transmitter that transmits a selected electromagnetic energy transmission for receipt by said helically-wound element.

21. The endovascular implant system of claim 20 further comprising:
a conductive composition carried in a surface of said wall portion thereby making said surface electrically conductive; and
circuitry within said wall portion coupling the at least one helically-wound element to said electrically conductive surface.

22. The endovascular implant system of claim 20 further comprising first and second spaced apart anode and cathode portions within a surface of said member and coupled to said at least one helically-wound element.
